(19) United States
(12) Patent Application Publication
(10) Pub. No.: US 2004/0220486 A1
(43) Pub. Date: Nov. 4, 2004

(54) Electrical and audio anatomy-signal sensor/coupler-adapter

(75) Inventors: Martin Baumer, Carlton, OR (US); Peter M. Galen, Portland, OR (US); Steven A. Mahoney, McMinnville, OR (US); Jagtar S. Saroya, Washougal, WA (US)

Correspondence Address: Robert D. Varitz, ROBERT D. VARITZ, P.C., 2007 S.E. Grant Street, Portland, OR, 97214, US

(73) Assignee: Inovise Medical, Inc.

(21) Appl. No.: 10/426,098

(22) Filed: Apr. 29, 2003

(51) Int. Cl. A61B 5/04

(52) U.S. Cl. 600/513; 600/528

(57) ABSTRACT

A releasably interconnectible, signal-communicative interface between an anatomical signal sensor and an associated receiving coupler-adapter. Through complementary male and female interconnection structures, this interface is designed to accommodate both a mechanical, and an electrical, releasably locked cross-connection between such a sensor and a coupler-adapter. Spring-biased cam-interengaging interface components, including a cam surface on the sensor, and a pair of relatively moveable opposed clamping arms on the coupler-adapter operate, during the act of connection, to draw a sensor projection into a connected condition within a socket provided in the coupler-adapter. A springy, electrically conductive component in the adapter plays both mechanical and electrical roles in relation to operation of the interface.

### BACKGROUND AND SUMMARY OF THE INVENTION

This invention relates to the collection, for review and monitoring purposes, of electrical and audio anatomical signals. In particular, it relates to combined mechanical and electrical interface structure which is provided for connecting a combined electrical and audio anatomy signal sensor with a coupler-adapter which is designed to convey, ultimately to external monitoring apparatus, sensor-acquired anatomical signals.

For the purpose of illustration and description herein, a preferred and best mode embodiment of the interface structure of the invention is described in the setting of collecting and conveying heart-produced anatomy signals, such as conventional ECG electrical signals, and related heart-produced audio signals, which are acquired by a sensor designed to perform both kinds of signal collection from a selected common anatomical site.

People involved in the field of cardiology are often interested in collecting and monitoring, preferably from (as nearly as possible) a common anatomical site, both ECG and related audio signals for subsequent conveyance to various kinds of external monitoring apparatus. In such a setting, and where a combined sensor is to be employed, there are also certain reasons making it desirable to provide a connective mechanical and electrical interface which utilizes an anatomical-site-positioned coupler-adapter between such a sensor and remote monitoring apparatus, with “outboard” electrical circuitry collaboratively employed by a connected sensor/coupler-adapter to function in various ways regarding, for example, signal collection, signal-processing, and/or outward signal conveyance to remote monitoring apparatus. Such an interface, cooperating with such circuitry which may be distributed both within such a sensor and within such a coupler-adapter, on opposite sides, so-to-speak, of the interface, makes possible the use of a wide variety of conventional external monitoring apparatus, with the added ability to enhance data-handling performance in certain ways without necessitating the modification of such pre-existing equipment.

The present invention is designed for use with these thoughts and considerations in mind. In particular, it is designed for use intermediate a “combined” sensor and an accommodating coupler-adapter, wherein each of these units may contain interface-uniteable, collaborative electrical circuit components which become cooperable on the occasion of a connection being established through the proposed interface.

Accordingly, proposed by the present invention, is a unique mechanical and electrical interface structure which is useable between a combined audio and electrical-anatomy signal sensor, and a compatible coupler-adapter, either or both of which may include, and, in accordance with capability that is offered by a preferred embodiment of the invention, do include, distributed electrical circuitry which performs certain outboard signal handling tasks that are thus accomplished essentially at the site of signal collection near the anatomy.

The various features and advantages that are offered by the present invention will become very fully apparent from the description which now follows, when that is read in conjunction with the accompanying drawings.

### DESCRIPTION OF THE DRAWINGS

FIG. 1 is a fragmentary, top, isometric view illustrating, in place on the anatomy (A), a combined audio and electrical anatomy-signal sensor, and a compatible and connected coupler-adapter, which connect and mate with one another operatively through an interface which is constructed in accordance with a preferred and best mode embodiment of the present invention.

FIG. 2 is a partially exploded, top, isometric view, somewhat like that presented in FIG. 1, showing the sensor and coupler-adapter of FIG. 1 disconnected from one another, separated from the anatomy, and partially displaying features of the interfacial mechanical and electrical structure provided according to the present invention.

FIG. 3 is an enlarged, fragmentary, bottom plan illustration of the underside (the anatomy-facing side) of the coupler-adapter of FIGS. 1 and 2. In FIG. 3, certain relevant dimensions (D₁, D₂) regarding two different operative conditions and positions of a pair of opposed, relatively moveable, spring-biased interface clamping arms which are included in the coupler-adapter of FIGS. 1 and 2, and which are exposed on the underside of this coupler-adapter, are highlighted.

FIG. 4 is a smaller-scale side elevation taken generally along the line 4-4 in FIG. 2. Dashed lines in FIG. 4 illustrate a special “shared-activity” component which is employed in the coupler-adapter illustrated herein.

FIG. 5 is an enlarged, fragmentary, cross-sectional view taken generally along the line 5-5 in FIG. 2, further illustrating details of the combined electrical and audio anatomical signal sensor of FIGS. 1 and 2 including interface structure (“companion” interface structure) which is constructed in accordance with the invention to cooperate with other “companion” interface structure that is present in the coupler-adapter of FIGS. 1-4.

FIG. 6, prepared on substantially the same scale which is used in FIG. 3, is a clamping-feature-emphasized, bottom plan view of the coupler-adapter of this invention, similar in many ways to the view presented in FIG. 3. This figure specifically illustrates, in a somewhat isolated fashion, the mentioned, opposed, spring-biased clamping arms which are present in the proposed coupler-adapter, with these arms, in FIG. 6, illustrated in the conditions wherein the dimension labeled D₁ in FIG. 3 defines the nominal diameter of a generally circular clamping nip region which is defined between these arms, and which is referred to herein as a “smaller-diameter nip region”.

FIG. 7, which is drawn on substantially the same scale employed in FIG. 6, is another clamping-feature-emphasized, bottom plan view of the coupler-adapter of this invention, also similar in many ways to the view presented in FIG. 3. This figure specifically illustrates, in a somewhat isolated fashion, the mentioned, opposed, spring-biased clamping arms which are present in the proposed coupler-adapter, with these arms, in FIG. 7, illustrated in the conditions wherein the dimension labeled D₂ in FIG. 3 defines the nominal diameter of a generally circular clamping nip region which is defined between these arms, and which is referred to herein as a “larger-diameter nip region”.

FIGS. 8 and 9 are stylized, inverted, cross-sectional views taken generally laterally centrally through FIGS. 6 and 7, respectively, with FIG. 8 isolating and showing a separation between the mentioned clamping arms that relates to dimension D₁ in FIG. 3, and with FIG. 9 isolating and showing a spacing between these same clamping arms relating to the dimension D₂ illustrated in FIG. 3.

FIGS. 10-12, inclusive, are fragmentary, cross-sectional views, taken generally from the same points of view presented in FIGS. 8 and 9, specifically showing the operation of interface reception, accommodation and clamping-in-place of the sensor of FIGS. 1, 2 and 5 in the drawings. In FIG. 10 the entry end of this sensor is shown just being received between the slightly more widely spaced gripping portions of the mentioned clamping arms. In FIG. 11 a camming action, which effects drawing-in of this sensor, is pictured, with that camming action taking place between the clamping arms herein and a portion of the entry end of the pictured sensor, all of which structure forms part of the interface structure of this invention. FIG. 12 illustrates full, clamped reception of this sensor by and between the clamping arms of the coupler-adapter illustrated herein.

FIG. 13 is an isolated, top, isometric view of what is referred to herein as a shared-activity component that is included in the interface structure of the invention and contained in the coupler-adapter shown in the drawings. This is the same component referred to above as being shown by dashed lines in FIG. 4.

### DETAILED DESCRIPTION OF THE INVENTION

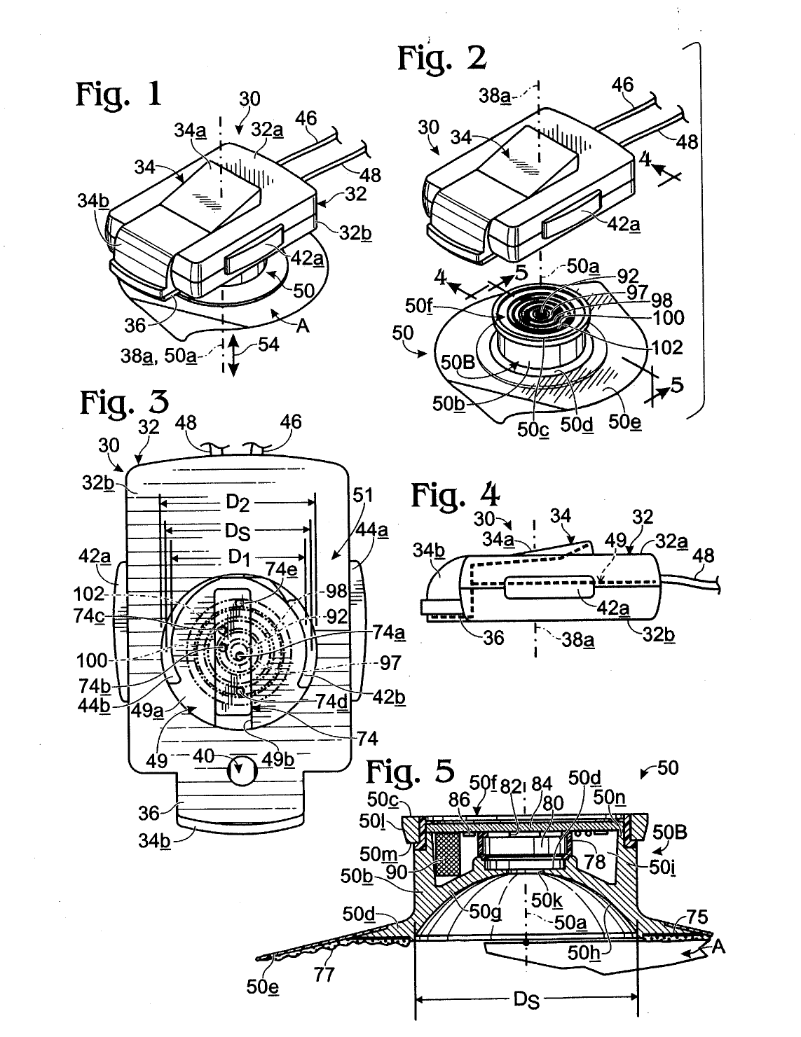

Turning now to the drawings, and referring first to FIGS. 1-9, inclusive, indicated generally at 30 is a coupler-adapter which includes interface structure constructed in accordance with a preferred and best-mode embodiment of the invention. For the sake of word economy hereinafter, coupler-adapter 30 will most often be referred to simply as adapter 30, or as the adapter. With respect to the specific embodiment of the interface structure of the present invention, this interface structure, and more particularly the portion of this interface structure which resides in the adapter structure, is illustrated in an adapter that is designed to receive and to accommodate, electrically and mechanically, plural, different types of anatomical signal sensors. And, while the subject interface structure is quite specific to a combined sensor like that shown in FIGS. 1, 2, 5 and 10-12, inclusive, this plural-sensor-handling capability will first be described and discussed just generally, in order to help with an understanding of the setting provided for the invention as shown herein.

Included, inter alia, in adapter 30 are (a) a housing, or body structure, 32, having upper and lower portions 32a, 32b, respectively, (b) a rocker paddle 34 with a thumb(or finger)-engaging portion 34a and a nose portion 34b, (c) a forwardly projecting lip 36 on which nose portion 34a is shown seated (in FIGS. 1, 2 and 4), (d) a pair of sensor reception sockets, or regions, 38, 40 which are exposed on lower housing portion 32b (see especially FIG. 3), and (e) a pair of spaced, laterally exposed squeeze buttons 42a, 44a which are disposed on laterally opposite sides of housing 32. Lower housing portion 32b defines the anatomy-facing “side” of adapter 30. Nose 34b is located at the front, or forward, end of the housing.

Shown fragmentarily, and extending from the rear end of adapter housing 32, are conductors 46, 48 which function to convey, to remote, external monitoring apparatus (not shown), electrical output signals that result ultimately from anatomical input signals that become collected by sensors coupled (connected) to the adapter.

Focusing attention for just a moment on FIG. 4, and including reference additionally to FIG. 13, shown generally at 49 is an electrically conductive, springy metal component which is suitably mounted within housing 32. This component functions herein as what is called a shared-activity component that plays, certain spring-biasing and electrical signal-conducting roles, with respect to attached sensors. Certain regions of, or portions within, component 49 play special roles, and these regions include a central, plate-like expanse 49a having a cut-out 49b, an L-shaped spring arm 49c which extends to one side of expanse 49a near cut-out 49b, a spring shelf 49d which extends into lip 36, and, as sub-portions of shelf 49d, a spiral-arm cantilever spring 49c with a “central”, end contact pad 49f, three angular, punch-formed ramps 49g, and a tab 49h. In relation to the interface structure of the present invention, the relevant portions of component 49 are central expanse 49a, cut-out 49b, and tab 49h.

Adapter 30 is designed herein to handle, in particular, three different kinds of sensors. One of these kinds (not involved with the present invention) takes the form of a conventional, somewhat circular, pad-like electrode (electrical-signal sensor only) which includes a central snap-prong-type connector that connects to adapter 30 on its anatomy-facing side through socket 40. Another sensor kind (also not involved with the present invention) takes the form of a conventional, elongate, rectangular strip electrode (also an electrical-signal sensor only) which connects in an alligator-grip fashion between rocker paddle nose 34b and lip 36. An alligator-grip region between these adapter components is opened and closed via suitable rocking of the rocker paddle utilizing thumb (finger)-engaging paddle portion 34a. Neither of these two sensor types is illustrated herein.

The third sensor type, however, is the kind which is involved with the present invention, and this is the type which is specifically illustrated generally at 50 in the drawings herein. With reference made particularly here to FIG. 1, in this figure, adapter 30 is shown in a condition receiving and accommodating (i.e. connected to) sensor 50, which is a combined audio and electrical anatomical (heart) signal sensor that includes, as has already been briefly mentioned, so-called companion interface structure constructed in accordance with the invention. In very general terms, the companion components of the interface structure of the present invention are shown at regions designated 51 in the drawings. Both the adapter and the sensor companion portions of this interface structure, as well as the interface structure as a whole, will, accordingly, hereinafter be designated commonly by the reference number 51.

As will be explained shortly, adapter 30 and sensor 50 are connectible and disconnectible, according to the structure and operation of interface 51, utilizing reception socket 38, and employing relative motion between the adapter and the sensor along a socket axis which is shown at 38a, and also along what (during connection, use, and disconnection) is then a coinciding sensor axis 50a. Connection and disconnection relative-motion is suggested in the drawings by double-headed arrow 54 (Arrow 54 is also employed as a single-headed arrow in certain other drawing figures.)

Before progressing the description of adapter 30, it will be useful here to describe a few of the externally visible features of sensor 50. Combined audio and electrical sensor 50 includes an elongate, somewhat spool-of-thread-shaped body, or body structure, 50B, with an elongate, central, cylindrical portion, or projection, 50b, a radially outwardly extending collar, or end, 50c and a radially outwardly extending skirt 50d formed at the upper and lower ends, respectively, of portion 50b. Also included are a flexible apron 50e, and a bull's eye pattern 50f of electrical conductors (central circular, and surrounding annular, traces) formed on the. top of the sensor body. Cylindrical portion 50b has an outside diameter (referred to herein as its “one” outside diameter) which is designated Dₛ herein (see FIGS. 5-12, inclusive). The undersides of skirt 50d and of apron 50e are coated appropriately with a conventional, sticky, electrically conductive hydrogel that functions to perform, among other things which will be discussed later, electrically conductive attachment to the anatomy. Collar 50c is also referred to herein as a region of revolution having “another” outside diameter which is larger than the above-mentioned “one” diameter. Conductor pattern 50f is also referred to herein as second electrical conductor structure. Body 50B is also referred to as male interconnection structure. More will be said later about conductor pattern 50f, and about certain other structural features of this combined electrical and audio sensor.

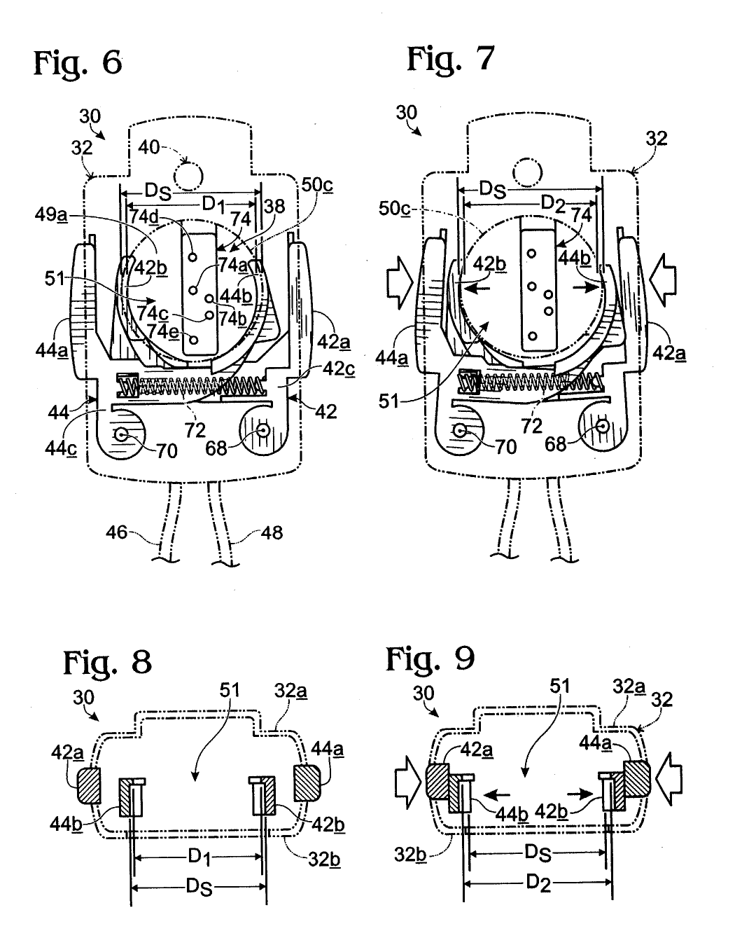

Continuing now a description of adapter 30, previously mentioned reception socket, or region 38, also referred to as a female interconnection structure, has a generally cylindrical configuration, as can be seen clearly in FIGS. 3, 6 and 7. With additional reference made now to FIGS. 10-12 in the drawings, one can see that laterally opposite sides of this generally cylindrical socket are somewhat defined by two, opposed, generally circularly curved (arcuate), relatively moveable clamping arms 42b, 44b which, along with previously mentioned squeeze buttons 42a, 44a, respectively, form component portions of two integrated, squeeze-button-operated clamping structures generally shown at 42, 44, respectively. (See especially FIGS. 6 and 7). Arms 42b, 44b collectively participate in defining sidewall structure in socket 38. Clamping arm 42a resides effectively on the opposite lateral side of housing 32 relative to the location of squeeze button 42a, and is joined to this squeeze button through an interconnecting base region 42c which is pivoted at 68 to appropriate other components in adapter 30. Similarly, arcuate clamping arm 44b resides on the opposite lateral side of housing 32 from its associated squeeze button 44a, and these two components are unified through a base region 44c which is pivoted appropriately at 70 on other components provided in adapter 30.

As can be seen especially well in FIGS. 3 and 6, the angular arcs subtended by clamping arms 42b, 44b are somewhat different. Very specifically, the arc subtended by arm 44b is greater than that subtended by arm 42b, and one of the reasons for this difference is to allow for base regions 42c, 44c, which extend overlappingly between these two arms, to enable a sufficient range of clamping-arm relative motion to accommodate convenient attachment and detachment of sensor 50.

Acting appropriately between clamping structures 42, 44 is a compression biasing spring 72 which urges the two clamping arms, and the two squeeze buttons associated with them, into the conditions and positions shown for them, respectively, in FIGS. 3, 6 and 8. In this condition of structures 42, 44, one can say that the confronting arcs of the two opposed clamping arms effectively define a generally circular nip region which has a nominal diameter that is shown at D₁ in FIGS. 3, 6 and 8. This nominal diameter D₁ is designed to be slightly smaller than the nominal outside diameter Dₛ of cylindrical portion 50b in sensor 50, and the nip region which it defines is referred to herein as a smaller-diameter nip region.

When squeeze buttons 42a, 44a are squeezed inwardly, as is indicated by the two, opposing, broad arrows that are pictured in FIGS. 7 and 9, these squeeze buttons and their associated clamping arms rock appropriately, against resistance presented by basing spring 72, about axes 68, 70, and move toward the respective positions that are shown for them in FIGS. 7 and 9. Here, one will notice that the two clamping arms have separated somewhat, and specifically have separated enough that they now effectively define a somewhat larger-diameter, enlarged nip region whose nominal diameter is represented in FIGS. 3, 7 and 9 at D₂. One will notice that diameter D₂ is slightly greater than the nominal diameter Dₛ of previously mentioned sensor cylindrical portion 50b.

In FIGS. 6 and 7, dimension Dₛ is related to a dash-double-dot circle which is designated 50c, and which represents generally the “footprint” which sensor collar 50c casts upon the outline of adapter 30 during insertion, use, and retraction of sensor 50 with respect to socket 38. In FIGS. 6 and 7, this dash-double-dot representation of sensor collar 50c is centered on previously mentioned socket axis 38a.

Referring now especially to FIGS. 3, 6, 7, 10-12, inclusive, and 13, forming what can be thought of as the base of socket 38 are (a) the central region, or portion, 49a in component 49, and (b) an electrical pin-connector block 74 which is shown herein including five individually spring-biased, outwardly projecting pin connectors, or pins, 74a, 74b, 74c, 74d and 74e (see particularly FIGS. 3, 6 and 7). These several pins, also referred to herein as first electrical conductor structure, project into socket 38, and connect appropriately both (at least some of them) with previously mentioned conductors 46, 48, and also with any signal processing electrical circuitry (not specifically shown) which may be contained and provided within housing 32 in sensor 30. At least one of these pins connects conductively at an appropriate location, such as at the location of a tab 49h in component 49 (see FIG. 13) with shared-activity component 49. Under normal circumstances when no sensor, such as sensor 50, is received in socket 38, the projecting pins that extend from pin block 74 extend into socket 38 as illustrated in FIG. 10 in the drawings. The just above mentioned first electrical conductor structure, along with the earlier mentioned second electrical conductor structure, constitute (when connected electrically as will shortly be explained) a signal-flow electrical connection.

Returning now to describe a bit more about sensor 50, and referring collectively to FIGS. 2, 3, 5 and 10-12, inclusive, sensor body 50, which functions in this sensor both as an electrical electrode, and as an acoustic sound-gathering component, is a unitary, integrated structure which includes, as was earlier mentioned, cylindrical central portion 50b, the upper part of which in the figures joins integrally with radially outwardly extending, rimming collar 50c, and the lower part of which joins integrally with radially outwardly extending, angular skirt 50d. Body 50B is a plastic molded structure which is molded from an appropriate, electrically conductive plastic material, and is formed as a somewhat hollow body of revolution which is symmetric with respect to previously mentioned axis 50a.

Extending as shown in body 50B, as seen in FIG. 5, is a central, compoundly curved, central bridging region 50g, the underside of which defines, and forms a downwardly facing, acoustic, sound-gathering cavity 50h. The upper side of region 50g, together with central portion 50b, define a component chamber 50i which includes a shallow, open, and generally cylindrical socket 50j. A passage 50k, which is cylindrical, and which is substantially centered on axis 50a, opens both to the base of socket 50j, and to the crown of cavity 50h. Cavity 50h is a volume of revolution centered on axis 50a.

Formed with collar 50c, which is also referred to as a locking shoulder herein, are an inclined, or angled, cam surface 50l which joins with a downwardly facing (in the figures) annular, shoulder locking surface 50m which extends radially inwardly from the cam surface to the outer surface of central cylindrical portion 50b.

The upper region of cylindrical portion 50b, radially inwardly relative to collar 50c, is recessed as shown, with the base of this recess being generally defined by a support shelf 50n which is annular in nature.

Appropriately formed on the underside of sensor body 50B, radially outwardly from cavity 50h, is a layer 75 (see FIG. 5) of an appropriate electrical conductor material, such as silver-silver-chloride. Apron 50e is suitably fastened to the top of skirt 50d. Applied to the undersides of layer 75 and of apron 50e is a conventional, sticky, electrically conductive hydrogel, or gel, material 77 (see FIG. 5) of the kind which is typically employed in anatomical ECG electrode-attaching applications. Gel 77 makes good electrical contact with layer 75, and through this layer, also makes good electrical contact with sensor body 50B.

Seated within the upper part of socket 50j in chamber 50i, and appropriately vibration-isolated by an elastomeric boot 78, is a microphone 80. Microphone 80 is aimed toward passage 50k, and is exposed through this passage to sounds that are gathered within cavity 50h. The upper side of microphone 80 is connected conductively, as shown, by conductive leads, such as lead 82 in FIG. 5, to suitable electrical conductor structure which is formed on the underside of a generally planar and circular, “overhead” circuit board 84 which rests on previously mentioned shelf 50n in the sensor body. Circuit board 84 carries electrical circuitry on its underside, in the form of various electrical circuit components, such as that shown at 86, which components are thus contained within chamber 50i in sensor body 50B.

Suitably supported within chamber 50i, to one side of microphone 80, is a battery 90. This battery is employed, as will shortly be explained, to provide energizing power for microphone 80, as well as for the electrical components that are carried on the underside of board 84 in the sensor.

As can be seen especially well in FIGS. 2 and 3, the upper side of circuit board 84 carries previously mentioned bull's-eye pattern 50f of electrical conductors. This pattern includes a central circular trace 92, and spaced radially outwardly therefrom, four, spaced, annular conductive traces 97, 98, 100 102. These conductive traces are connected through appropriate, and otherwise conventional, vias (not specifically shown) that extend through the body of circuit board 84 to connect with battery 90 and with microphone 80.

Finally, locking various components in place with respect to sensor body 50B is an electrically conductive washer 104. Washer 104 establishes an electrically conductive connection betweens sensor body 50B and conductive trace 102 in conductor pattern 50f. With washer 104 in place, therefore, there exists an electrically conductive path extending from gel 77, through layer 75, sensor body 50B, washer 104, and annular conductive trace 102, and through certain ones of the vias mentioned above, to connect appropriately with battery 90 and with the electrical components contained on circuit board 84, as well as with microphone 80.

Returning now to adapter 30, pins 74a-74e, inclusive, herein are appropriately positioned within the confines of socket 38, whereby, as one will now see, they may each come into contact with a different one of the various conductive traces furnished in conductor pattern 50f. Very specifically, and as can be seen in FIG. 3, pins 74a, 74b, 74c, 74d, 74e are positioned to engage traces 92, 97, 98, 100, 102, respectively, in sensor 50 when the sensor and adapter are connected. One will note from this pin-trace relationship that when adapter 30 and sensor 50 are connected, the “stability” of the electrical connections established between the pins and traces is indifferent to any relative rotation which may take place between the adapter and sensor about coincident axes 38a, 50a.

When it is desired to connect sensor 50 to adapter 30 at the location of socket 38, squeeze buttons 42a, 44a, are squeezed toward one another against the action of biasing spring 72. This action places clamping arms 42a, 42b generally in the positions shown for them in FIGS. 7 and 9. In this condition of these clamping arms, the larger-diameter, generally circular nip region defined between them is large enough to permit the diameter of collar 50c to pass inwardly beyond the arms and into socket 38. Initial entry of collar 50c between the thus spaced clamping arms is shown generally in FIG. 10.

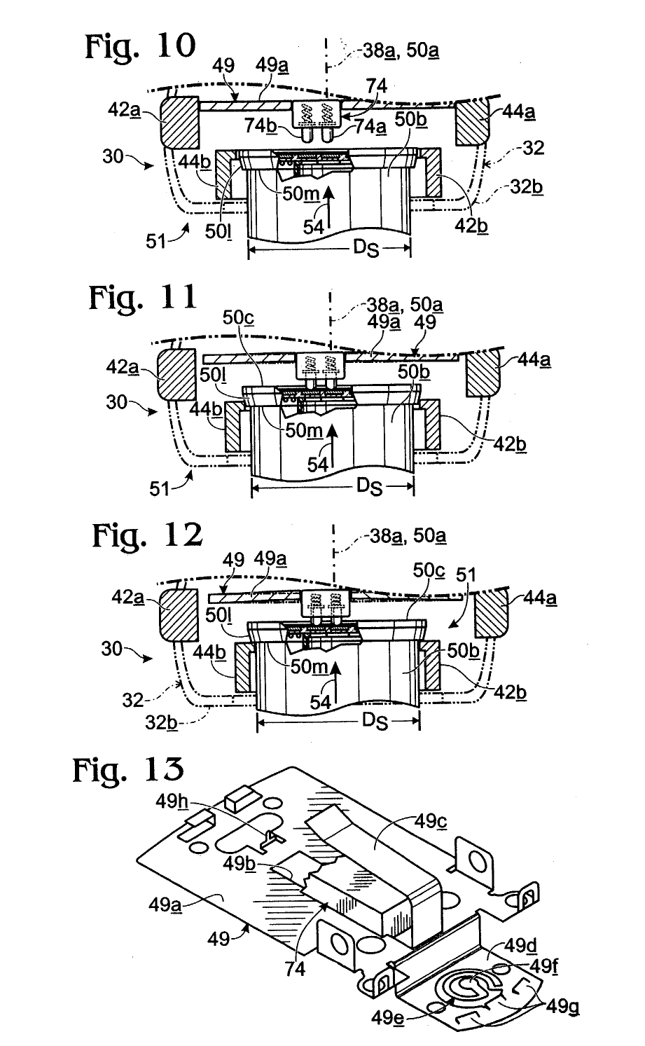

With continued inward slight movement of sensor 50, and with relaxation of squeeze pressure on buttons 42a, 44a, the closest opposing surfaces in clamping arms 42b, 44b come into edge contact with cam surface 50l, and this situation is illustrated clearly in FIG. 11. The regions of the clamping arms which so engage surface 50l are referred to herein as cam-engaging surfaces.

If one now simply completely releases the squeeze buttons, biasing spring 72 drives the clamping arms toward the positions shown for them in FIGS. 6 and 8, and because of the engagement just previously described between these clamping arms and cam surface 50l, such inward spring-biased driving action functions to draw sensor 50 inwardly along axes 38a, 50a into socket 38. Very specifically, the sensor is drawn into socket 38 to a point where the clamping arms can snap inwardly under the influence of spring 72 to produce a kind of positive anti-retraction lock by engagement with collar shoulder 50m. This condition is illustrated in FIG. 12. In this connected condition between the adapter and the sensor, the sensor is held in socket 38 by what is referred to herein as a multi-orthogonal-axial clamping action which includes force vectors generally furnished along traditional X, Y and Z orthogonal axes.

Under circumstances with camming action taking place, as is illustrated in FIG. 11, sensor 50 will have entered socket 38 far enough to create engagements between the projecting pins in block 74 and the conductive traces in conductor pattern 50f. Such engagement causes the pins to shift inwardly into block 74 against the respective biasing of their biasing springs, and this can be seen to be taking place in FIG. 11. There is thus a situation now where electrical contact is made between the conductive traces in pattern 50f and the buttons projecting from block 74. Additionally, a mechanical biasing action begins to occur, whereby the pins that project from block 74 exert a downward, or outwardly axially rejecting, force against the entry end of sensor 50. With block 74 anchored to the central portion 49a of shared-activity component 49, a slight springy deflection takes place in component portion 49a, and this is suggested by comparing the dash-double-dot and solid outline conditions illustrated in FIGS. 11 and 12 for this central portion of component 49.

Accordingly, component 49, cooperating with the biasing springs that are provided for the projecting pins in block 74, and also cooperating with these pins themselves, urges sensor 50 axially in a manner wherein, when the state of affairs pictured in FIG. 12 is achieved, these cooperating biasing structures serve to urge the sensor, and very specifically shoulder 50m, against the clamping arms to help to engage, hold and stabilize sensor 50 with respect to adapter 30. It is in relation to this biasing activity which has just been described that component 49 plays a role in mechanical spring-biasing with respect to an attached condition existing between adapter 30 and a sensor like sensor 50. In addition, because at least one of the pins which projects from block 74 makes contact with one of the electrical traces on the entry end of sensor 50, and because this pin is one which is electrically connected to component 49, a signal-management electrical flow path is created wherein component 49 plays a signal-communication role in the operation of an attached sensor 50.

When it is desired to decouple sensor 50 from adapter 30, this is done very simply by once again squeezing inwardly on the squeeze buttons thus to open up the spacing between the clamping arms, and then drawing outwardly on sensor 50 to disconnect it.

The novel interface of this invention is thus described. Through it, a coupler-adapter and a sensor of the types illustrated and described, easily are connected and disconnected both mechanically and electrically. During connection, camming action works to draw the projecting body structure of the sensor into the reception socket provided in the coupler-adapter. Locking action takes place to hold these two structures together against inadvertent disconnection. Rotational symmetry that associates inter-engaging electrically conducting pins and surface traces, enables a connected coupler-adapter and sensor to be rotationally indifferent with regard to stability of an interface-created electrical signal connection.

The adapter and sensor may each conveniently be provided with internal circuitry, including an energizing battery, which become operatively cooperative during a connection—which connection is designed to be effective to create circuitry energization by the battery. Such enabled circuitry can furnish convenient signal processing/handling at the site of a nearby association with the anatomy, and may thus enable certain selected kinds of useful signal processing without requiring that existing remote signal-monitoring apparatus be modified.

Variations and modifications of the invention are certainly possible, and may be recognized by persons generally skilled in the art. All such variations and modifications are deemed to be within the scope of this invention.

We claim:

1. A releasably interconnectible connective and signal-communicative interface between a sensor for gathering anatomical, electrical and audio signals and a coupler-adapter for receiving the sensor, and for communicating such gathered signals to external structure, and where the sensor and the coupler-adapter each includes body structure, said interface comprising
a socket in the body structure of the coupler-adapter, having a base, and sidewall structure extending from said base including a pair of spring-biased, selectively adjustable, opposed, relatively moveable clamping arms,
first electrical conductor structure provided in said coupler-adapter and located adjacent said base,
an elongate projection in the body structure of the sensor, removably receivable within said socket to create a connected condition between the sensor and the coupler-adapter, in which condition, said clamping arms, under the influence of the mentioned spring-biasing which is provided for them, releasably grip and hold said projection within said socket, said projection including an end adapted to face said socket base with the projection disposed within said socket, and
second electrical conductor structure formed on said projection adjacent its said end, and adapted to establish, through contact with said first electrical conductor structure, a signal-flow electrical connection under circumstances with said projection gripped and held by said clamping arms.

2. The interface of claim 1, wherein said projection includes a long axis, and a generally cylindrical portion substantially axially symmetrically centered on said axis and having one outside diameter, said end takes the form of a region of revolution also substantially symmetrically centered on said axis and possessing another outside diameter which is greater than said one diameter, and said clamping arms are shaped, and nominally spring-biased, to define a generally cylindrical nip region having a nominal diameter which is smaller than said one diameter, with these arms being selectively adjustable, via relative separating motion between them against the associated spring-biasing influence, to enlarge said nip region to possess an enlarged diameter which is greater than said other diameter.

3. The interface of claim 2, wherein the coupler-adapter further includes, operatively mounted on its said body structure, opposed and relatively moveable, manually operable squeeze buttons that are operatively connected to said clamping arms in a manner whereby squeezing of the buttons to move them relatively toward one another produces relative motion between said clamping arms in a way that urges the arms, against spring-biasing action, to enlarge said nip region.

4. The interface of claim 2, wherein said projection end is formed with a radially outwardly facing, angled cam surface, said clamping arms are formed each with a cam-engaging surface whereby, during insertion of said projection into said socket, any engagement, radial with respect to said axis, then occurring between said cam surface and said cam-engaging surface under the influence of spring-biasing action which tends to close said nip region toward its said nominal diameter, produces an axially-directed force vector which tends to draw said projection axially inwardly into said socket to advance said projection end toward said base.

5. The interface of claim 4, wherein said projection end is further formed with a locking shoulder with a locking surface which joins at an angle with said cam surface to produce selectively-defeatable, anti-disconnection locking between said projection and said clamping arms under conditions wherein said projection has entered said socket by a certain axial distance, and said clamping arms have closed upon one another, due to spring-biasing action, to place the clamping arms in contact with said projection's generally cylindrical portion.

6. The interface of claim 5, wherein (a) said first electrical conductor structure takes the form of plural elongate, exposed, axially spring-biased pins which extend and are biased toward the direction from which the sensor projection end moves relative to the socket base during the act of connection between the sensor and the coupler-adapter, (b) said second electrical conductor structure takes the form of a pattern of conductors formed on the far extent of said projection end, and (c), an established releasable interconnection between the sensor and the coupler-adapter, under circumstances with said projection gripped and held by said clamping arms, results in said pins being disposed in respective, spring-biased-resistance yield conditions wherein they establish a spring-biased, forced conductive connection with conductors in said pattern of conductors.

7. The interface of claim 1, wherein (a) said first electrical conductor structure takes the form of plural elongate, exposed, axially spring-biased pins which extend and are biased toward the direction from which the sensor projection end moves relative to the socket base during the act of connection between the sensor and the coupler-adapter, (b) said second electrical conductor structure takes the form of a pattern of conductors formed on the far extent of said projection end, and (c), an established releaseable interconnection existing between the sensor and the coupler-adapter, under circumstances with said projection gripped and held by said clamping arms, results in said pins being disposed in respective, spring-biased-resistance yield conditions wherein they establish a spring-biased, forced conductive connection with conductors in said pattern of conductors.

8. The interface of claim 6, wherein conductors in said pattern of conductors include annular conductor traces which are radially symmetric with respect to said axis, and said pins include at least certain pins that are adapted to engage with said traces whereby, with an operative releasable interconnection existing between the sensor and the coupler-adapter, stability of conductive engagement between said at least certain pins and said traces is indifferent to the rotational disposition of the sensor relative to the coupler-adapter with reference to said axis.

9. The interface of claim 1, wherein the sensor is adapted to collect both electrical and audio anatomical signals which are heart-related.

10. A releasably interconnectable, connective and signal-communicative interface between a sensor for gathering anatomical electrical and audio signals and a coupler-adapter for receiving the sensor, and for communicating such gathered signals to external structure, where the sensor and the coupler-adapter each includes body structure, said interface comprising
male and female interconnection structure disposed within the respective body structures of the sensor and the coupler-adapter, and
adjustable clamping structure provided in said female interconnection structure, operable releasably to produce multi-orthogonal-axial, spring-biased, clamped locking between the sensor and the coupler-adapter.

11. The interface of claim 10, wherein said clamping structure includes electrical conductor structure through which communicated electrical signals flow.

12. The interface of claim 10, wherein connection and disconnection of said interconnection structures takes place along a linear interconnection axis with respect to which each interconnection structure has a defined connection/disconnection topography, and said multi-orthogonal-axial clamped locking is characterized by force vectors directed along traditional X. Y and Z orthogonal axes, one of which is substantially coincident with said interconnection axis.

13. The interface of claim 12, wherein said clamping structure includes electrical conductor structure through which communicated electrical signals flow, with said conductor structure defining a flow path for such communicated signals which generally parallels said linear interconnection axis.
